(19) United States
(12) Patent Application Publication
(10) Pub. No.: US 2009/0196839 A1
(43) Pub. Date: Aug. 6, 2009

(54) DISPERSION OF POLYMER PARTICLES, COMPOSITION COMPRISING SAME AND COSMETIC TREATMENT METHOD

(76) Inventor: Celine FARCET, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/362,862

(22) Filed: Jan. 30, 2009

Related U.S. Application Data
(60) Provisional application No. 61/064,022, filed on Feb. 11, 2008.

(30) Foreign Application Priority Data
Feb. 1, 2008 (FR) 0850636

(51) Int. Cl. A61K 8/81; A61K 31/765; A61K 31/785; A61Q 5/00; A61Q 17/04; A61Q 5/04; A61Q 1/02; A61Q 1/06; A61Q 1/12; A61Q 3/00; A61K 8/25; A61Q 5/08; A61Q 19/10; A61Q 5/02; A61Q 5/10; A61Q 5/12

(52) U.S. Cl. 424/59; 424/78.31; 424/78.35; 424/78.17; 424/78.18; 424/78.02; 424/78.03; 424/70.16; 424/70.17; 424/70.2; 424/70.7; 424/63; 424/64; 424/69; 424/61; 510/122; 510/119; 510/159; 510/158; 8/405; 424/62

(57) ABSTRACT

Dispersions of particles of surface-stabilized polymer, in a nonaqueous medium, in which the polymer contains 5% to 50% by weight of ethylenic monomers which can contain a PEG (polyethylene glycol) part. Dispersions can be used in cosmetic or pharmaceutical compositions such as lipstick or hair. The composition can be used to treat keratin materials.

This application claims benefit of U.S. Provisional Application No. 61/064,022, filed Feb. 11, 2007, the contents of which are incorporated herein by reference. This application also claims benefit of priority under 35 U.S.C. § 119 to French Patent Application No. FR 0850636, filed Feb. 1, 2007, the contents of which are also incorporated herein by reference.

The present disclosure relates to a novel dispersion of particles of polymers, dispersed in a nonaqueous medium, and also to the compositions, such as cosmetic or pharmaceutical compositions, comprising said dispersion.

It is known practice to use, in cosmetics, dispersions of polymer particles, generally of nanometric size, in organic media, and for example, through EP749747, nonaqueous dispersions of poly(methyl(meth)acrylate) particles, in a non-volatile paraffin oil or in isododecane, for example. However, these dispersions do not make it possible to obtain optimal cosmetic properties, for example, in terms of glide, comfort, staying power and feel, and further for example, softness.

Surprisingly, novel dispersions of polymer particles, stabilized by stabilizing agents, in nonaqueous media, which make it possible to provide the desired cosmetic properties (feel, softness, glide) have been discovered.

A subject of the disclosure is therefore a dispersion of particles of at least one ethylenic polymer surface-stabilized with a stabilizing agent, in a nonaqueous medium comprising at least one nonaqueous compound, which is liquid at 25° C., having an overall solubility parameter according to the Hansen solubility space of less than or equal to 20 (MPa)^{1/2}, or a mixture of such compounds; characterized in that said ethylenic polymer comprises from 5% to 50% (excluded) (i.e. less than) by weight of monomer of formula (I) as defined hereinafter, alone or as a mixture, relative to the total weight of monomers.

A subject of the disclosure is also a cosmetic or pharmaceutical composition comprising, in a cosmetically or pharmaceutically acceptable medium, at least one dispersion as defined above.

It is now possible to prepare polymers that can be readily carried given that the dispersions have viscosities that are not very high, thereby facilitating their use in cosmetic compositions.

In the makeup field, these compositions may provide increased comfort properties, for example an improved glide, and further for example in a moist environment. Furthermore, the polymers may have a resistance and staying power with respect to external attacks, for example considerable rubbing (meals). The comfort and the staying power may therefore be improved. In the case of lipsticks, the use of saliva on the lips may make it possible to generate a swelling effect, or volume, which has a fullness effect; the moist film thus created at the surface protects the deposit and gives a “wetted” sheen.

In the haircare field, these compositions may confer volume and shape retention on the head of hair. In addition, these dispersions or compositions may make it possible to obtain, in addition to the styling effects and the shape retention, a soft and non-sticky feel, softness, and also easy disentangling and smoothing of the hair, in a dry and/or wet medium.

The polymer particles may be very small in size, for example, nanometric, which is not the case with, for example, other types of particles, such as microspheres, the diameter of which is generally greater than 1 micron. However, a large size of the order of a micron may have the drawback of giving the particles a certain visibility to the eye, when they are in a composition and when they are applied to the skin, and also may lead to poor stability of the composition, for example, over time. Thus, the dispersions according to the disclosure make it possible to obtain stable compositions which may also be transparent, translucent or opaque, as desired, depending on the size of the polymer particles which are dispersed therein.

Furthermore, the dispersions according to the disclosure are in oily media, which is favored in the makeup field; however, polymers comprising PEG (polyethylene glycol) units were up until now generally used in aqueous solution or dispersion, and therefore more difficult to formulate in the makeup field.

The dispersions according to the disclosure are therefore constituted of particles, generally spherical particles, of at least one ethylenic polymer, surface-stabilized with a stabilizing agent, in a nonaqueous medium.

The dispersions according to the disclosure may for example be in the form of nanoparticles of polymers in a stable dispersion in a nonaqueous medium. The nanoparticles, for example have a size of from 5 to 600 nm, such as from 10 to 500 nm, further such as from 15 to 450 nm, given that, above approximately 600 nm, the particle dispersions may become much less stable.

These particles may remain in the form of elementary particles, without forming agglomerates, when they are in a dispersion in said nonaqueous media.

The term “ethylenic” polymer is intended to mean a polymer obtained by polymerization of at least two monomers, which may be identical or different, comprising an ethylenic unsaturation. Said ethylenic polymer may be chosen by those skilled in the art, as a function of its properties, according to the desired subsequent use for the composition. These polymers may be, for example, crosslinked.

The polymers according to the disclosure may be linear, branched, grafted, or even star, homopolymers or copolymers. They may be random or alternating. For example, they are linear random copolymers.

The ethylenic polymer according to the invention comprises 5% (included) (i.e. greater than or equal to) to 50% (excluded) (i.e. less than) by weight of hydrophilic monomer, or of a mixture of such monomers, relative to the total weight of monomers.

The ethylenic polymer, for example comprises from 7 to 49% by weight, such as from 8% to 45% by weight, further such as from 10% to 40% by weight, and even further such as from 15% to 35% by weight, of hydrophilic monomer, alone or as a mixture, relative to the total weight of monomers, which can also be referenced as starting monomers.

The hydrophilic monomers, for the purpose of the invention, are chosen from, alone or as a mixture, the monomers of formula (I):

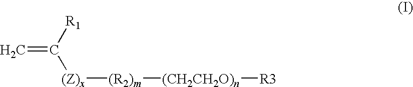

in which:
R1 is a hydrogen atom or a linear or branched CₚH₂ₚ₊₁ hydrocarbon-based radical, with p being an integer from 1 to 12;
Z is a divalent group chosen from —COO—, —CONH—, —CONCH₃—, —OCO—, —O—, —SO₂—, —CO—O—CO—, and —CO—CH₂—CO—;
x is 0 or 1;
R2 is a linear, branched, cyclic, optionally aromatic, or alicyclic, optionally aromatic, saturated or unsaturated, substituted or unsubstituted, divalent carbon-based radical containing from 1 to 30 carbon atoms, which may comprise from 1 to 18 heteroatoms chosen from O, N, S, F, Si, and P;
m is 0 or 1;
n is an integer from 3 to 300;
R3 is a hydrogen atom or a linear, branched, cyclic, optionally aromatic, or alicyclic, optionally aromatic, saturated or unsaturated, unsubstituted or substituted, carbon-based radical containing from 1 to 30 carbon atoms, which may comprise 1 to 20 heteroatoms chosen from O, N, S, F, Si, and P;
   and/or at least one salt thereof.

For example, R1 may represent a methyl, ethyl, propyl, or butyl radical. Further for example, R1 represents hydrogen or a methyl radical.

For example, Z represents —COO— or —CONH—, such as —COO—.

For example, x is equal to 1.

In the R2 radical, the heteroatom(s), when it (they) is (are) present, may be intercalated in the chain or said R2 radical, or else said R2 radical may be substituted with one or more groups comprising them, such as hydroxyl, amino (NH2, NHR′ or NR′R″ with R′ and R″, which may be identical or different, representing a linear or branched C₁-C₂₂ alkyl, such as methyl or ethyl, —CF₃, —CN, —SO₃H, or —COOH.

For example, R2 may comprise an —O—, —N(R)— or —CO— group, or a combination thereof, and for example —O—CO—O—, —CO—O—, —N(R)CO—, —O—CO—NR—, or —NR—CO—NR—, with R representing H or a linear or branched C₁-C₂₂ alkyl, optionally comprising 1 to 12 heteroatoms chosen from O, N, S, F, Cl, Br, Si, and P.

For example, R2 may be:
an alkylene radical containing 1 to 20 carbon atoms, such as methylene, ethylene, n-propylene, isopropylene, n-butylene, isobutylene, tert-butylene, pentylene, isopentylene, n-hexylene, isohexylene, heptylene, isoheptylene, n-octylene, isooctylene, nonylene, isononylene, decylene, isodecylene, n-dodecylene, isododecylene, tridecylene, n-tetradecylene, hexadecylene, n-octadecylene, docosanylene, or arachinylene;
a substituted or unsubstituted cycloalkylene radical containing 5 to 10 carbon atoms, such as cyclopentylene, cyclohexylene, cycloheptylene, cyclooctylene, cyclononylene or cyclodecylene;
a phenylene radical —C₆H₄-(ortho, meta or para) unsubstituted or substituted with a C₁-C₁₂ alkyl radical optionally comprising 1 to 18 heteroatoms chosen from O, N, S, F, Si and P;
a benzylene radical —C₆H₄—CH₂— unsubstituted or substituted with a C₁-C₁₂ alkyl radical optionally comprising 1 to 18 heteroatoms chosen from O, N, S, F, Si and P;
a radical of formula —CH₂—O—CO—O—, —CH₂—CH₂—O—CO—O—, —CH₂—CO—O—, —CH₂—CH₂—CO—O—, —CH₂—O—CO—NH—, —CH₂—CH₂—O—CO—NH—; —CH₂—NH—CO—NH—, —CH₂—CH₂—NH—CO—NH—; —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″), —CH₂—CH(NR′R″)—, —CH₂—CH₂—CH₂—NR′—, —CH₂—CH₂—CH₂—O—; —CH₂—CH₂—CHR′—O— with R′ and R″ representing a linear or branched C₁-C₂₂ alkyl optionally comprising 1 to 12 heteroatoms chosen from O, N, S, F, Si, and P;
or a mixture of these radicals.

As a further example, R2 may be:
an alkylene radical containing 1 to 20 carbon atoms, for example methylene, ethylene, n-propylene, n-butylene, n-hexylene, n-octylene, n-dodecylene, or n-octadecylene;
a phenylene radical —C₆H₄-(ortho, meta or para) optionally substituted with a C₁-C₁₂ alkyl radical optionally comprising 1 to 18 heteroatoms chosen from O, N, S, F, Si, and P; or
a benzylene radical —C₆H₄—CH₂— optionally substituted with a C₁-C₁₂ alkyl radical optionally comprising 1 to 18 heteroatoms chosen from O, N, S, F, Si, and P.

For example, n is from 5 to 200, such as from 7 to 100, or further such as from 9 to 50.

For example, R3 is a hydrogen atom; a phenyl radical unsubstituted or substituted with a C₁-C₁₂ alkyl radical optionally comprising 1 to 20 heteroatoms chosen from O, N, S, F, Si, and P; a C₁-C₃₀, such as C₁-C₂₂, or further such as C₂-C₁₆, alkyl radical optionally comprising 1 to 18 heteroatoms chosen from O, N, S, F, Si and P; or a C₃-C₁₂, such as C₄-C₈, or further such as C₅-C₆, cycloalkyl radical optionally comprising 1 to 18 heteroatoms chosen from O, N, S, F, Si, and P.

These alkyl, cycloalkyl or phenyl radicals may comprise for example one or more functions chosen from the following functions:

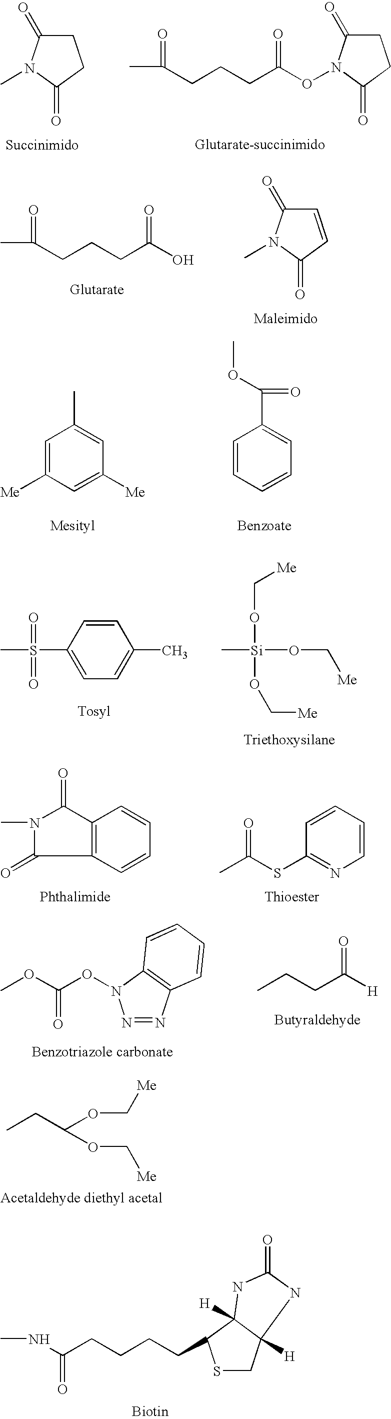

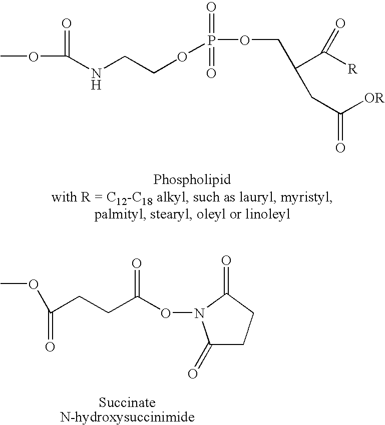

and/or also chosen from —SO₃H, —COOH, —PO₄, —NR5R6 or —N⁺R5R6R7, with R5, R6 and R7, independently of one another, chosen from H or linear, branched or cyclic C₁-C₁₈ alkyl, such as methyl, optionally comprising one or more heteroatoms or else bearing protective groups such as t-butyloxycarbonyl (also called BOC) or 9-fluorenylmethoxycarbonyl (also called FmoC).

Among the R3 radicals, mention may be made of methyl, ethyl, propyl, benzyl, ethylhexyl, lauryl, stearyl and behenyl (—(CH₂)₂₁—CH₃) chains, and also fluoroalkyl chains, for instance heptadecafluorooctyl sulphonylaminoethyl CF₃—(CF₂)₇—SO₂—N(C₂H₅)—CH₂—CH₂; or else —CH₂—CH₂—CN, succinimido, maleimido, mesityl, tosyl, triethoxysilane or phthalimide chains.

For example, the monomers of formula (I) are such that:
R1=H or methyl,
Z represents COO,
x=1
m=0,
n=7 to 100,
R3 is chosen from a hydrogen atom; a phenyl radical optionally substituted with a C₁-C₁₂ alkyl radical; and a C₁-C₃₀, such as C₁-C₂₂, or further such as C₂-C₁₆, alkyl radical.

Among representative monomers of formula (I) mention may be made of:
poly(ethylene glycol) (meth)acrylate in which R1 is H or methyl; Z is —COO—, x=l, m=0 and R3=H;
methyl poly(ethylene glycol) (meth)acrylate, also called methoxy poly(ethylene glycol) (meth)acrylate, in which R1 is H or methyl, Z is —COO—, x=l, m=0 and R3=methyl;
alkyl poly(ethylene glycol) (meth)acrylate in which R1 is H or methyl, Z is —COO—, x=l, m=0 and R3=alkyl;
phenyl poly(ethylene glycol) (meth)acrylates, also called poly(ethylene glycol) phenyl ether (meth)acrylate, in which R1 is H or methyl, Z is —COO—, x=l, m=0 and R3=phenyl.

Examples of commercially available monomers are:
CD 350 (methoxy poly(ethylene glycol 350) methacrylate) and CD 550 (methoxy poly(ethylene glycol 550) methacrylate), supplied by SARTOMER Chemicals;
M90G (methoxy poly(ethylene glycol) (9 repeating units) methacrylate) and M230G (methoxy poly(ethylene glycol) (23 repeating units) methacrylate) available from Shin-Nakamura Chemicals;
methoxy poly(ethylene glycol) methacrylates having an average molecular weight of 300, 475 or 1100, available from Sigma-Aldrich;
methoxy poly(ethylene glycol) acrylate having an average molecular weight of 426, available from Sigma-Aldrich;
the methoxy poly(ethylene glycol) methacrylates available from LAPORTE under the trade names: MPEG 350, MPEG 550, S10W, S20W, or from Cognis under the name BISOMER;
the poly(ethylene glycol) monomethyl ether, mono(succinimidyl succinate) esters having an average molecular weight of 1900 or 5000, from Polysciences;
behenyl poly(ethylene glycol PEG-25) methacrylate, available from Rhodia, under the name SIPOMER BEM;
poly(ethylene glycol) phenyl ether acrylates having an average molecular weight of 236, 280 or 324, available form Aldrich;
the methoxy polyethylene glycol 5000 2-(vinyl sulphonyl)ethyl ether commercially available from Fluka;
the poly(ethylene glycol) ethyl ether methacrylate available from Aldrich;
the poly(ethylene glycol) 8000, 4000, 2000 methacrylates from Monomer & Polymer Dajac laboratories.

Further representative monomers of formula (I) are chosen from poly(ethylene glycol) (meth)acrylates and alkyl poly(ethylene glycol) (meth)acrylates, such as methyl poly(ethylene glycol) methacrylates.

The ethylenic polymer according to the invention may of course comprise a single monomer of formula (I) or a mixture of such monomers.

The polymers present in the dispersion are therefore derived from the polymerization of one or more monomers of formula (I) and of one or more additional monomers, which are therefore present in a proportion of 50% (included)(i.e. equal to or greater than) to 95% by weight, such as from 51% to 97% by weight, or further such as from 55% to 98% by weight, and even further such as from 60% to 90% by weight, and even further such as from 65% to 85% by weight, relative to the total weight of monomers.

Among the additional monomers that can be used in the context of the present invention, mention may be made, alone or as a mixture, of the monomers of formula (II), and salts thereof:

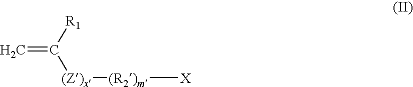

in which:
R1 is a hydrogen atom or a linear or branched CₚH₂ₚ₊₁ hydrocarbon-based radical, with p being an integer from 1 to 12;
   For example, R1 may represent a methyl, ethyl, propyl or butyl radical. Further for example R1 may represent hydrogen or a methyl radical;
Z′ is a divalent group chosen from —COO— and —CONH—;
x′ is 0 or 1, for example 1;
R′2 is a linear, branched, cyclic, optionally aromatic, or alicyclic, optionally aromatic, saturated or unsaturated, divalent carbon-based radical containing from 1 to 30 carbon atoms, which may comprise 1 to 18 heteroatoms chosen from O, N, S, F, Si, and P;

For example, R12 may be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄-(ortho, meta or para) optionally substituted with a C₁-C₁₂ alkyl radical optionally comprising 1 to 18 heteroatoms chosen from N, O, S, F, Si and/or P; or else a benzylene radical —C₆H₄—CH₂—, optionally substituted with a C₁-C₁₂ alkyl radical optionally comprising 1 to 18 heteroatoms chosen from O, N, S, F, Si and P;
or a mixture of these radicals;
m′ is 0 or 1;
X is chosen from —H, —COOH, —SO₃H, —OSO₃H, —PO₃H₂ and —OPO₃H₂.

Among the monomers of formula (II) mention may be made, alone or as a mixture, of (meth)acrylamide, (meth)acrylic acid, crotonic acid, itaconic acid, fumaric acid, maleic acid, diacrylic acid, dimethylfumaric acid, citraconic acid, acrylamidopropanesulphonic acid, 2-acrylamido-2-methylpropanesulphonic acid, styrenesulphonic acid, vinylbenzoic acid, vinylphosphoric acid, vinylsulphonic acid, vinylbenzenesulphonic acid, acrylamidoglycolic acid of formula CH₂═CH—CONHCH(OH)COOH, vinylphosphonic acid; sulphopropyl (meth)acrylate (CH₂═C(CH₃)CO₂(CH₂)₃SO₃H), sulphoethyl(meth)acrylate, vinylmethylsulphone, 2-(methacryloyloxy)ethyl phosphate of formula CH₂═C(CH₃)COOC₂H₄OP(O)(OH)₂; and also salts thereof; and mixtures thereof.

The additional monomers may also be chosen from the following monomers, alone or as a mixture:
(i) (meth)acrylic acid esters of formula CH₂═CHCOOR4 or CH₂═C(CH₃)COOR4 with R4 representing a linear, cyclic, or branched, saturated or unsaturated hydrocarbon-based radical containing 1 to 32 carbon atoms, optionally comprising intercalated one or more heteroatoms chosen from O, N, and S, and/or optionally substituted with one or more substituents chosen from —OH and halogen atoms (Cl, Br, I, and F).
, the carbon-based chain R4 may be:
a C₁-C₃₂ alkyl group;
a C₃ to C₈ cycloalkyl group;
a C₆ to C₂₀ aryl group;
a C₇ to C₃₀ aralkyl group (C₁ to C₄ alkyl group);
a heterocyclic group comprising 4 to 12 ring members containing one or more heteroatoms chosen from O, N and S;
a heterocycloalkyl group (C₁ to C₄ alkyl) such as a furfuryl group; it being possible for said alkyl, cycloalkyl, aryl, aralkyl, heterocyclic, or heterocycloalkyl groups to comprise intercalated one or more heteroatoms chosen from O, N, S, and/or to be substituted with one or more substituents chosen from the hydroxyl group, halogen atoms, and linear or branched C₁-C₄ alkyl groups in which are optionally intercalated one or more heteroatoms chosen from O, N, S, and P, and/or it being possible, in addition, for said alkyl groups to be optionally substituted with one or more substituents chosen from the hydroxyl group and halogen atoms (Cl, Br, I, and F).

Mention may thus be made of methyl, ethyl, propyl, n-butyl, isobutyl, 2-ethylhexyl, octyl, isooctyl, isodecyl, decyl, lauryl, tridecyl, dodecyl, myristyl, cetyl, palmityl, stearyl, behenyl and oleyl (meth)acrylates; tert-butyl, t-butylcyclohexyl, t-butylbenzyl, furfuryl and isobornyl (meth)acrylates; and 2-hydroxyethyl, ethoxyethyl, 2-methoxyethyl, and hydroxypropyl (meth)acrylates.
(ii) (meth)acrylamides of formula CH₂═CHCONR5R15 or CH₂═C(CH₃)CONR5R′5 in which R5 and R′5, which may be identical or different, represent a hydrogen atom or a linear, cyclic or branched, saturated or unsaturated, optionally aromatic, hydrocarbon-based radical containing 7 to 28 carbon atoms, optionally comprising intercalated one or more heteroatoms chosen from O, N and S, and/or optionally substituted with one or more substituents chosen from —OH and halogen atoms (Cl, Br, I, and F).

For example, the carbon-based chain R5 and/or R′5 may be:
a C₇-C₂₈ alkyl group;
a C₇ to C₈ cycloalkyl group;
a C₇ to C₂₀ aryl group;
a C₇ to C₂₈ aralkyl group (C₁ to C₄ alkyl group);
a heterocyclic group comprising 4 to 12 ring members containing one or more heteroatoms chosen from O, N and S;
a heterocycloalkyl group (C₁ to C₄ alkyl) such as a furfuryl group;
   it being possible for said alkyl, cycloalkyl, aryl, aralkyl, heterocyclic or heterocycloalkyl groups to comprise intercalated one or more heteroatoms chosen from O, N, S, and/or to be substituted with one or more substituents chosen from the hydroxyl group, halogen atoms and linear or branched C₁-C₄ alkyl groups in which are optionally intercalated one or more heteroatoms chosen from O, N, S and P, and/or it being possible, in addition, for said alkyl groups to be optionally substituted with one or more substituents chosen from the hydroxyl group and halogen atoms (Cl, Br, I, and F).

Examples of such monomers are N-tert-butyl(meth)acrylamide, N-butyl(meth)acrylamide, N-isobutyl(meth)acrylamide, N-propylmethacrylamide, N-isopropylmethacrylamide, N-hexyl(meth)acrylamide, N-2-ethylhexyl(meth)acrylamide, N-octyl(meth)acrylamide, N-isooctyl(meth)acrylamide, N-nonyl(meth)acrylamide, N-undecyl(meth)acrylamide, N-dodecyl(meth)acrylamide, N-tridecyl(meth)acrylamide, N-tetradecyl(meth)acrylamide, N-hexadecyl(meth)acrylamide, N-paimityl(meth)acrylamide, N-octadecyl(meth)acrylamide, N-docosanoyl(meth)acrylamide, N-octadecenoyl(meth)acrylamide, N-cyclohexyl(meth)acrylamide, N-phenyl(meth)acrylamide, N-isobornyl(meth)acrylamide, N-benzyl(meth)acrylamide, N,N-dibutyl(meth)acrylamide, and N,N-diethylaminopropyl(meth)acrylamide;
(iii) vinyl esters of formula CH₂═CH—OCO—R6 with R6 representing a linear or branched, saturated or unsaturated, hydrocarbon-based radical containing 1 to 12 carbon atoms, among which mention may be made of vinyl acetate, vinyl butyrate (or butanoate), vinyl ethylhexanoate, vinyl neononanoate, vinyl neododecanoate, vinyl propionate, vinyl hexanoate, vinyl ethylhexanoate, vinyl octanoate, vinyl decanoate, vinyl pivalate, vinyl palmitate, vinyl stearate, vinyl cyclohexanoate, vinyl benzoate and vinyl 4-tert-butylbenzoate;
(iv) vinyl ethers of formula CH₂═CHOR7 with R7 representing a linear or branched, saturated or unsaturated, hydrocarbon-based radical containing 2 to 12 carbon atoms; among which mention may be made of ethyl vinyl ether, ethyl hexyl vinyl ether, butyl vinyl ether, isobutyl vinyl ether, cyclohexyl vinyl ether, octyl vinyl ether, decyl vinyl ether, dodecyl vinyl ether, hexadecyl vinyl ether, and octadecyl vinyl ether;
(v) vinyl compounds of formula CH₂═CHR8 in which R8 is:
a hydroxyl group;
a linear or branched alkyl group containing 1 to 25 carbon atoms, in which is (are) optionally intercalated one or more heteroatoms chosen from O, N, S, and P; it being possible for said alkyl group, in addition, to be optionally substituted with one or more substituents chosen from —OH and halogen atoms (Cl, Br, I, and F);
a C₃ to C₈ cycloalkyl group such as cyclohexane;
a C₆ to C₂₀ aryl group such as phenyl;
a C₇ to C₃₀ aralkyl group (C₁ to C₄ alkyl group) such as 2-phenylethyl or benzyl;
a heterocyclic group comprising 4 to 12 ring members containing one or more heteroatoms chosen from O, N and S;
a heterocycloalkyl group (alkyl containing 1 to 4 carbon atoms), such as furfuryl, furfurylmethyl or tetrahydrofurfurylmethyl;
   it being possible for said cycloalkyl, aryl, aralkyl, heterocyclic or heterocycloalkyl groups to be optionally substituted with one or more substituents chosen from the hydroxyl group, halogen atoms and linear or branched C₁-C₄ alkyl groups in which is (are) optionally intercalated one or more heteroatoms chosen from O, N, S, and P, it being possible, in addition, for said alkyl groups to be optionally substituted with one or more substituents chosen from —OH and halogen atoms (Cl, Br, I, and F).

Examples of vinyl monomers are vinylcyclohexane, styrene, vinylcaprolactam, methylstyrene; 4-tert-butylstyrene, 4-acetoxystyrene; 4-methoxystyrene, 3-methylstyrene; 4-methylstyrene, 2-chlorostyrene, 3-chlorostyrene, 4-chlorostyrene, dimethylstyrene; 2,6-dichlorostyrene; 2,4-dimethylstyrene; 2,5-dimethylstyrene; 3,5-ethoxystyrene; 2,4-ethoxy-styrene, vinylbutyral; vinyl chloride; vinyl formal; vinylidene chloride, vinylidene fluoride, 2-vinylnaphthalene; N-methylmaleimide; 1-octene, 1-butene; chlorobutadiene, chlorotrifluoroethylene; cis-isoprene, trans-isoprene; 1-octadecene; butadiene; hexadecene, eicosene, and 4-fluorostyrene.

Among the additional monomers mention may be made of the following monomers, and mixtures thereof:
the (meth)acrylic acid esters with R4 as C₁-C₁₈, and such as methyl(meth)acrylate, ethyl (meth)acrylate, propyl(meth)acrylate, n-butyl(meth)acrylate, isobutyl(meth)acrylate, 2-ethylhexyl(meth)acrylate, tert-butyl(meth)acrylate, isooctyl (meth)acrylate, decyl (meth)acrylate, myristyl (meth)acrylate, stearyl (meth)acrylate or isobornyl (meth)acrylate;
styrene, ethyl hexyl vinyl ether, dodecyl vinyl ether, vinyl hexanoate;
acrylic acid and methacrylic acid.

When it comprises acid functions, the polymer according to the invention may be completely or partially neutralized with an organic base, for example a primary, secondary or tertiary amine or alkylamine, it being possible for the amine to comprise or not comprise substituents (hydroxyl) and/or one or more nitrogen and/or oxygen atoms. Mention may be made, for example, of 2-amino-2-methylpropanol, triethylamine, butylamine, triethanolamine, 2-(dimethylamino)propanol, lysine, 3-(dimethylamino)propylamine, cyclohexylamine, N-octylamine, N-dodecylamine and N-octadecylamine.

When it comprises basic functions, the polymer according to the invention may be completely or partially neutralized with organic acids which may comprise one or more carboxylic, sulphonic or phosphonic groups. They may be linear, branched or cyclic aliphatic acids or alternatively aromatic acids. Mention may be made, for example, of acetic acid, alpha-hydroxyethanoic acid, alpha-hydroxyoctanoic acid, alpha-hydroxycaprylic acid, ascorbic acid, benzoic acid, behenic acid, capric acid, caproic acid, caprylic acid, citric acid, dodecylbenzenesulphonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric acid (or 2-butyloctanoic acid), isomyristic acid (or 2-hexyloctanoic acid), isoarachidic acid (or 2-octyldodecanoic acid), isolignoceric acid (or 2-decyltetradecanoic acid), lactic acid, lauric acid, malic acid, myristic acid, oleic acid, palmitic acid, propionic acid, sebacic acid, stearic acid, tartaric acid, terephthalic acid, trimesic acid, undecylenic acid, propylbetain, cocoamidopropylbetain, and mixtures thereof.

It is possible to neutralize the monomers having a neutralizable acid and/or amine function before they are polymerized, or else to neutralize the polymer once formed. For example, the polymer is neutralized after formation thereof.

The polymer may have only one glass transition temperature.

However, it may have several glass transition temperatures, such as two Tg; in this case, further such as, the lowest Tg is below +20° C.

The polymers that can be used in the context of the present invention may have a number-average molecular weight (Mn) of from 2000 to 1 000 000, such as from 3000 to 800 000, and further such as from 4000 to 500 000, or even further such as from 10 000 to 300 000.

The dispersion of particles of polymers according to the invention therefore comprises a nonaqueous medium in which said particles are dispersed.

This nonaqueous medium is constituted of at least one nonaqueous compound, which is liquid at 25° C., having an overall solubility parameter according to the Hansen solubility space of less than or equal to 20 (MPa)^{1/2}, or a mixture of such compounds.

The overall solubility parameter δ according to the Hansen solubility space is defined in the article “Solubility parameter values” by Grulke, in the book “Polymer Handbook” 3rd edition, chapter VII, pages 519-559, by the equation:

δ=(d_{D}²+d_{P}²+d_{H}²)^{1/2}

in which:
d_{D} characterizes the London dispersion forces derived from the formation of dipoles induced during molecular impacts,
d_{P} characterizes the Debye interaction forces between permanent dipoles,
d_{H} characterizes the specific interaction forces (of the type hydrogen bonds, acid/base, donor/acceptor, etc.)

The definition of the solvents in the three-dimensional solubility space according to Hansen is described in the article by Hansen: “The three dimensional solubility parameters” J. Paint Technol. 39, 105 (1967).

Among the nonaqueous liquid compounds having an overall solubility parameter according to the Hansen solubility space of less than or equal to 20 (MPa)^{1/2}, mention may be made of liquid fatty substances, such as oils, which can be chosen from optionally branched, natural or synthetic carbon-based, hydrocarbon-based, fluoro and/or silicone oils, alone or as a mixture.

Among these oils, mention may be made of plant oils formed by fatty acid esters of polyols, such as triglycerides, further such as sunflower oil, sesame oil, or rapeseed oil, or esters derived from 6 to 20 carbon atoms acids or alcohols, even further such as esters of formula RCOOR′ in which R represents the residue of a higher fatty acid containing from 7 to 19 carbon atoms and R′ represents a hydrocarbon-based chain containing from 3 to 20 carbon atoms, such as palmitates, adipates, myristates, and benzoates, further such as diisopropyl adipate and isopropyl myristate.

Mention may also be made of hydrocarbons, such as paraffin oil, liquid petroleum jelly, or hydrogenated polyisobutylene, C₈-C₁₆ isoparaffins and volatile isoparaffins such as isododecane or “ISOPARs”.

Mention may also be made of silicone oils such as polydimethylsiloxanes and polymethylphenylsiloxanes, optionally substituted with optionally fluorinated aliphatic and/or aromatic groups, or with functional groups such as hydroxyl, thiol and/or amine groups; and volatile, for example cyclic or linear silicone oils, such as cyclodimethylsiloxanes, cyclophenylmethylsiloxanes, and linear dimethylsiloxanes, among which mention may be made of linear dodecamethylpentasiloxane (L5), octamethylcyclotetrasiloxane, decamethylcyclopentasiloxane, hexadecamethylcyclohexasiloxane, heptamethylhexyltrisiloxane, and heptamethyloctyltrisiloxane.

Mention may also be made of the solvents, alone or as a mixture, chosen from linear, branched or cyclic esters containing 6 to 30 carbon atoms, ethers containing 6 to 30 carbon atoms, and ketones containing 6 to 30 carbon atoms.

Among the nonaqueous compounds that can be used, mention may also be made of monoalcohols having an overall solubility parameter according to the Hansen solubility space of less than or equal to 20 (MPa)^{1/2}, i.e. aliphatic fatty monoalcohols containing at least 6 carbon atoms, such as 6 to 32 carbon atoms, the hydrocarbon-based chain not comprising a substituting group. As monoalcohols according to the invention, mention may be made of oleyl alcohol, decanol, dodecanol, octadecanol, and linoleyl alcohol.

In an embodiment, the nonaqueous medium comprises volatile, for example cyclic or linear, silicone oils such as cyclodimethylsiloxanes and linear dimethylsiloxanes, and/or esters of formula RCOOR′ in which R represents the residue of a higher fatty acid containing from 7 to 19 carbon atoms and R′ represents a hydrocarbon-based chain containing from 3 to 20 carbon atoms, such as palmitates, adipates, myristates and benzoates, such as diisopropyl adipate and isopropyl myristate; and also mixtures thereof.

In another embodiment, the nonaqueous medium comprises hydrocarbons, such as paraffin oil, liquid petroleum jelly, or hydrogenated polyisobutylene, C₈-C₁₆ isoparaffins and volatile isoparaffins such as isododecane or “ISOPARs”.

The choice of the nonaqueous medium can be made readily by those skilled in the art according to the nature of the monomers constituting the polymer and/or to the nature of the stabilizing agent.

The polymer dispersion may be produced as described in document EP-A-749747. In general, the polymerization may be carried out as dispersion polymerization, i.e. by precipitation of the polymer during formation, with protection of the formed particles using a stabilizing agent.

A mixture comprising the starting monomers and also a radical initiator can therefore be prepared. This mixture is dissolved in a synthesis solvent.

The monomers are soluble in the reaction medium, whereas the polymer is not soluble therein. Over the course of the polymerization, the polymer will precipitate and be stabilized by the stabilizing agent present. Particles of polymers surface-protected by the stabilizing agent are thus obtained.

The polymerization can be carried out directly in the nonaqueous medium, which can thus also play the role of synthesis solvent.

It is also possible to carry out the polymerization in a synthesis solvent, and then to subsequently perform a solvent exchange, replacing the synthesis solvent with the nonaqueous medium.

Thus, when the nonaqueous medium chosen is a non-volatile hydrocarbon-based or silicone oil, the polymerization can be carried out in an apolar organic solvent (synthesis solvent) and then the non-volatile oil (which must be miscible with said synthesis solvent) can be added and the synthesis solvent can be selectively distilled.

A synthesis solvent may therefore be chosen such that the starting monomers and the radical initiator are soluble therein, and the particles of polymer obtained are insoluble therein such that they precipitate therein as they form. For example, the synthesis solvent may be chosen from alkanes such as heptane, isododecane, or cyclohexane.

When the nonaqueous medium chosen is a volatile hydrocarbon-based or silicone oil, the polymerization can be carried out directly in said oil, which therefore also plays the role of synthesis solvent. The monomers may, for example, also be soluble therein, as should the radical initiator, and the polymer obtained should be insoluble therein.

The monomers are, for example, present in the synthesis solvent, before polymerization, in a proportion of 5-80% by weight. All the monomers may be present in the solvent before the beginning of the reaction, or a part of the monomers may be added as the polymerization reaction progresses.

The radical initiator may, for example, be an azo or peroxide compound, such as azobisisobutyronitrile or tert-butyl peroxy-2-ethylhexanoate.

The polymer particles are surface-stabilized.

In an embodiment, the particles can be surface-stabilized during the course of the polymerization, by virtue of a stabilizing agent which, for example, may be a sequential polymer, a grafted polymer and/or a random polymer, alone or as a blend. Stabilization can be carried out by any known means and, for example, by polymerization in the presence of the stabilizing agent.

The stabilizing agent may be present in the mixture at the start of the polymerization. However, it is also possible to add it continuously, for example when monomers are also being added continuously.

In another embodiment, the polymer can be synthesized in a synthesis solvent and then dispersed in a nonaqueous dispersion medium by addition of the dispersing agent, and the synthesis solvent can be evaporated.

Use may be made of 0.1% to 30% by weight of the stabilizing agent relative to the weight of the starting mixture of monomers, such as from 1% to 25% by weight, further such as from 2% to 20% by weight, or even further such as from 3% to 15% by weight.

When a grafted and/or sequential polymer is used as stabilizer, the synthesis solvent is chosen in such a way that at least a portion of the grafts or sequences of said stabilizing polymer is soluble in said solvent, the other portion of the grafts or sequences not being soluble therein. The stabilizing polymer used during the polymerization should be soluble or dispersible in the synthesis solvent. Furthermore, a stabilizing agent comprising a portion (sequences, grafts, or the like) exhibiting a degree of affinity for the polymer formed during the polymerization may be chosen.

When a random polymer is used as stabilizing agent, it is chosen in such a way that it has a sufficient amount of groups rendering it soluble in the synthesis solvent envisaged.

Among the grafted polymers, mention may be made of silicone polymers grafted with a hydrocarbon-based chain and hydrocarbon-based polymers grafted with a silicone chain.

Also suitable are grafted copolymers having, for example, an insoluble backbone of polyacrylic type with soluble grafts of poly(12-hydroxystearic acid) type.

As stabilizing polymer, mention may also be made of:
(a) grafted- or sequential-block copolymers comprising at least one block of polyorganosiloxane type and at least one block of a polymer (i) resulting from radical polymerization or (ii) resulting from polycondensation, such as of polyether, polyester or polyamide type, or a blend thereof, it being possible for said copolymer to comprise fluorinated entities.

As grafted- or sequential-block copolymers comprising at least one block of polyorganosiloxane type and at least one block of a radical polymer, mention may be made of grafted copolymers of acrylic/silicone type which can be employed, for example, when the nonaqueous medium is a silicone medium.

When the grafted- or sequential-block copolymers comprise at least one block of polyorganosiloxane type and at least one polyether block, the polyorganopolysiloxane block may, for example, be a polydimethylsiloxane or, further for example, a poly(C₂-C₁₈)alkylmethylsiloxane; the polyether block may be a C₂-C₁₈ polyalkylene, such as polyoxyethylene and/or polyoxypropylene. Use may thus be made of dimethicone copolyols or else (C₂-C₁₈)alkyl methicone copolyols, which are optionally crosslinked. Use may, for example, be made of the dimethicone copolyol sold under the name “Dow Corning 3225C” by the company Dow Corning, or the lauryl methicone copolyol sold under the name “Dow Corning Q2-5200” by the company Dow Corning.

Mention may also be made of lauryl dimethicone copolyol crosspolymer, for example KSG31 or KSG32 from Shin-Etsu, cetyl dimethicone copolyol, such as DMC 3071 from GE, and dimethicone copolyol PPG-3 oleyl ether, such as KF-6026 from Shin-Etsu;
(b) grafted- or sequential-block copolymers of C₁-C₄ alkyl (meth)acrylates and of C₈-C₃₀ alkyl (meth)acrylates. Mention may be made of the stearyl methacrylate/methyl methacrylate copolymer;
(c) grafted- or sequential-block copolymers comprising at least one block resulting from the polymerization of ethylenic monomer, comprising one or more optionally conjugated ethylenic bonds, and/or, for example, of dienes; and at least one block of polymer resulting from radical polymerization other than dienes, such as resulting from vinyl monomer, (meth)acrylic monomer, or (meth)acrylamide monomer, or of a polyether, of a polyester or of a polyamide, or blends thereof.

For example, use may be made of copolymers comprising at least one block resulting from the polymerization of at least one ethylenic monomer, comprising one or more optionally conjugated ethylenic bonds, such as ethylene, butadiene or isoprene, and at least one block of a styrene polymer. When the ethylenic monomer comprises several optionally conjugated ethylenic bonds, the residual ethylenic unsaturations after the polymerization are generally hydrogenated. Thus, in a known way, the polymerization of isoprene results, after hydrogenation, in the formation of an ethylene-propylene block, and the polymerization of butadiene results, after hydrogenation, in the formation of an ethylene-butylene block. Among these sequential copolymers, mention may be made of copolymers of “diblock” or “triblock” type, of the polystyrene/polyisoprene or polystyrene/polybutadiene type, such as those sold under the name “Luvitol HSB” by BASF, of the polystyrene/copoly(ethylene-propylene) type, such as those sold under the name “Kraton” by Shell Chemical Co, or else of the polystyrene/copoly(ethylene-butylene) type.

As grafted- or sequential-block copolymers comprising at least one block resulting from the polymerization of at least one ethylenic monomer, such as ethylene or isobutylene, and at least one block of an acrylic polymer such as methyl methacrylate, mention may be made of poly(methyl methacrylate)/polyisobutylene bi- or trisequential copolymers or grafted copolymers comprising a poly(methyl methacrylate) backbone and comprising polyisobutylene grafts.

As grafted- or sequential-block copolymers comprising at least one block resulting from the polymerization of at least one ethylenic monomer and at least one block of a polyether, such as a polyoxy(C₂-C₁₈)alkylene, further such as polyoxyethylene and/or polyoxypropylene, mention may be made of polyoxyethylene/polybutadiene or polyoxyethylene/polyisobutylene bi- or trisequential copolymers.

Use may also be made, as stabilizing agent, of compounds such as:
(d) alkyl dimethicones in which the alkyl group comprises 6 to 32 carbon atoms, such as lauryl methicone and stearyl methicone, further such as Si tec LDM 3107 from ISP, cetyl dimethicone, even further such as Abil Wax 9801, behenoxy dimethicone, and even further such as Abil 5440 from Goldschmidt;
(e) dimethiconol esters of formula:

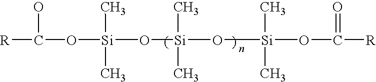

in which R is an alkyl radical containing 6 to 32 carbon atoms, such as dimethiconol behenate, and further such as the products Ultrabee from Noveon and Pecosil DB from Phoenix Chemical;
(f) alkylamidoamines containing, for example, 6 to 60 carbon atoms, further for example 12 to 50, such as behenamidopropyldimethylamine and further such as Catemol 220 from Phoenix Chemical, of formula:

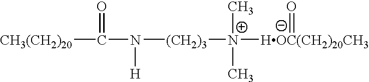

(g) copolymers comprising at least one polyorganosiloxane part and fluorinated groups, such as fluorinated silicones or fluorosilicones which can be represented by the formula:

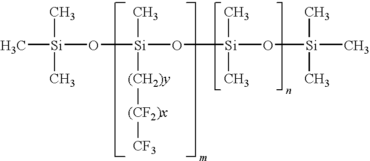

in which x is an integer from 3 to 12, such as from 5 to 10, and further such as x=8; y is an integer from 2 to 6, such as from 2 to 3; and m and n are such that the molecular weight of the compound is from 5000 to 15 000;
and for example perfluorononyl dimethicones, such as those sold under the name Pecosil FSH-150 and 300 or Pecosil FSL-150 and 300 by Phoenix Chemical.

When the synthesis solvent is apolar, it is possible to choose, as stabilizing agent, a polymer providing as complete a covering as possible of the particles, several chains of stabilizing polymers then being adsorbed onto one particle of polymer obtained by polymerization.

In this case, for example, it is possible to use as stabilizing agent, either a grafted polymer or a sequential polymer, so as to have better interfacial activity. This is because the sequences or grafts which are insoluble in the synthesis solvent provide a more voluminous covering at the surface of the particles.

When the liquid synthesis solvent comprises at least one silicone oil, the stabilizing agent may be chosen from the group constituted of grafted- or sequential-block copolymers comprising at least one block of polyorganosiloxane type and at least one block of a radical polymer or of a polyether or of a polyester, such as polyoxypropylenated and/or oxyethylenated blocks.

When the liquid synthesis solvent comprises at least one hydrocarbon-based oil, such as isoparaffin, the stabilizing agent is, for example, chosen from grafted- or sequential-block copolymers comprising at least one block resulting from the polymerization of at least one ethylenic monomer, comprising one or more optionally conjugated ethylenic bonds, such as ethylene, butadiene or isoprene, and at least one block of a styrene polymer; and, for example, the stabilizing agent is a “diblock” or “triblock” copolymer of the polystyrene/polyisoprene, polystyrenelpolybutadiene, polystyrene/copoly(ethylene-propylene), or polystyrene/copoly(ethylene-butylene) type.

It is possible to add, to the dispersion of polymers, a plasticizer so as to lower the Tg of the polymers used. The plasticizer may be chosen from the plasticizers customarily used in the field of application, such as from compounds capable of being solvents for the polymer. The plasticizer may be integrated during the synthesis or added once the synthesis has been carried out.

The dispersions obtained according to the invention may then be used in a composition, such as a cosmetic or pharmaceutical composition, which comprises, moreover, a cosmetically or pharmaceutically acceptable medium.

Depending on the application envisaged, use may be made of dispersions of film-forming or non-film-forming polymers in a nonaqeuous medium comprising volatile or non-volatile oils.

The composition may then comprise, depending on the application envisaged, the constituents customary in this type of composition.

Among these constituents, mention may be made of waxes, oils, gums and/or pasty fatty substances of plant, animal, mineral or synthetic origin, or even silicone origin, and mixtures thereof.

Among the waxes that may be present in the composition according to the invention, mention may be made, alone or as a mixture, of hydrocarbon-based waxes such as beeswax; carnauba wax, candelilla wax, ouricury wax or Japan wax, cork fiber or sugar cane waxes; paraffin waxes or lignite waxes; microcrystalline waxes; lanolin wax; montan wax; ozokerites; polyethylene waxes; waxes obtained by Fischer-Tropsch synthesis; hydrogenated oils, fatty esters and glycerides which are solid at 25° C. Use may also be made of silicone waxes, among which mention may be made of alkyl derivatives, alkoxy derivatives and/or esters of polymethylsiloxane.

The composition according to the invention may also comprise carbon-based, hydrocarbon-based, fluorinated and/or silicone oils of mineral, animal, plant or synthetic origin, alone or as a mixture, in so far as they form a homogeneous and stable mixture and in so far as they are compatible with the use envisaged. Among the oils that may be present in the composition according to the invention, mention may be made of, alone or as a mixture, hydrocarbon-based oils such as paraffin oil or liquid petroleum jelly; perhydrosqualene; arara oil; sweet almond oil; calophyllum oil, palm oil, castor oil, avocado oil, jojoba oil, olive oil or cereal germ oil; esters of lanolic acid, of oleic acid, of lauric acid or of stearic acid; alcohols such as oleyl alcohol, linoleyl alcohol or linolenyl alcohol, isostearyl alcohol or octyl dodecanol. Mention may also be made of silicone oils, such as PDMSs, which are optionally phenylated, such as phenyl trimethicones. Use may also be made of volatile oils, such as cyclotetradimethylsiloxane, cyclopentadimethylsiloxane, cyclohexadimethylsiloxane, methylhexyldimethylsiloxane, hexamethyldisiloxane or isoparaffins.

The composition according to the invention may also comprise one or more dyestuffs chosen from pulverulent compounds and/or fat-soluble or water-soluble dyes, for example in a proportion of from 0.01% to 70% of the total weight of the composition.

The pulverulent compounds may be chosen from pigments and/or pearlescent agents and/or the fillers commonly used in cosmetic or pharmaceutical compositions. Advantageously, the pulverulent compounds represent from 0.1% to 50% of the total weight of the composition, such as from 1% to 40%.

The pigments may be white or colored, inorganic and/or organic, and interferential or noninterferential. Among the inorganic pigments, mention may be made of titanium dioxide, which is optionally surface-treated, zirconium or cerium oxides, and also iron or chromium oxides, manganese violet, ultramarine blue, chromium hydrate and ferric blue. Among the organic pigments, mention may be made of carbon black, pigments of D & C type and lakes based on cochineal carmine, barium, strontium, calcium or aluminum.

The pearlescent pigments may be chosen from white pearlescent pigments, such as mica coated with titanium or with bismuth oxychloride, colored pearlescent pigments, such as titanium mica with iron oxides, titanium mica with, such as, ferric blue or chromium oxide, titanium mica with an organic pigment of the abovementioned type, and also pearlescent pigments based on bismuth oxychloride.

The fillers may be inorganic or organic and lamellar or spherical. Mention may be made of talc, mica, silica, kaolin, nylon and polyethylene powders, poly-β-alanine and polyethylene powders, Teflon, lauroyllysine, starch, boron nitride, tetrafluoroethylene polymer powders, hollow microspheres such as Expancel (Nobel Industrie), Polytrap (Dow Corning) and silicone resin microbeads (Tospearls from Toshiba, for example), precipitated calcium carbonate, magnesium carbonate, basic magnesium carbonate, hydroxyapatite, hollow silica microspheres (Silica Beads from Maprecos), glass or ceramic microcapsules, metal soaps derived from organic carboxylic acids containing from 8 to 22 carbon atoms, such as from 12 to 18 carbon atoms, for example zinc stearate, magnesium stearate, lithium stearate, zinc laurate, or magnesium myristate.

The fat-soluble dyes are, for example, Sudan red, DC Red 17, DC Green 6, β-carotene, soybean oil, Sudan brown, DC Yellow 11, DC Violet 2, DC Orange 5 or quinoline yellow. They may represent from 0 to 20%, such as from 0.01% to 20%, of the weight of the compositions, and further such as from 0.1% to 6%. The water-soluble dyes are, for example, beetroot juice or methylene blue and can represent up to 6% of the total weight of the composition.

The composition may also comprise surfactants, which may be chosen from cationic, anionic, amphoteric and nonionic surfactants, and mixtures thereof. These surfactants may be present in a proportion of from 0.01% to 30% by weight, such as from 0.05% to 20% by weight, relative to the total weight of the composition.

The composition may additionally comprise any additive normally used in the cosmetics field, such as antioxidants, fragrances, essential oils, preservatives, cosmetic active agents, moisturizing agents, vitamins, essential fatty acids, ceramides, sunscreens, polymers, thickeners or gelling agents. Of course, those skilled in the art will take care to choose this or these additive(s) and/or the amount thereof in such a way that the advantageous properties of the composition according to the invention are not, or are not substantially, impaired by the envisaged addition.

The compositions according to the invention may be in any form acceptable and conventional for a cosmetic or pharmaceutical composition.

The composition according to the invention may be in the form of a suspension, a dispersion, for example of oil in water by virtue of vesicles; an optionally thickened, or even gelled, oily solution; an oil-in-water, water-in-oil or multiple emulsion; a gel or a foam; an oily or emulsified gel; a dispersion of vesicles, such as lipid vesicles; a two-phase or multiphase lotion; a spray; of a lotion, of a cream, of an ointment, of a soft paste, of a salve, of a cast or molded solid, such as cast or molded as a wand or in a dish, or else of a compacted solid.

Those skilled in the art may choose the appropriate galenical form, and also the method for preparing same, on the basis of their general knowledge, taking into account, on the one hand, the nature of the constituents used, for example the solubility thereof in the carrier, and, on the other hand, the application envisaged for the composition.

The cosmetic composition according to the invention may be in the form of a product for caring for and/or making up the skin of the body or face, the lips and the hair, of an antisun or self-tanning product, or even of a hair product.

It has advantageous application in the haircare field, such as for hairstyle form retention or hair shaping. The hair compositions may be shampoos, gels, hair-setting lotions, blow-drying lotions or fixing and styling compositions, such as lacquers, mousses or sprays. The lotions may be packaged in various forms, such as in vaporizers, pump-action sprays or aerosol containers, in order to provide for application of the composition in vaporized form or in the form of a mousse.

The compositions in accordance with the invention may be used for washing or treating keratin materials such as the hair, the skin, the eyelashes, the eyebrows, the nails, the lips or the scalp, and further such as the hair.

The compositions according to the invention may be detergent compositions, such as shampoos, shower gels and bubble baths.

The compositions of the invention may also be in the form of a rinse-out or leave-in conditioner, of permanent-waving, hair straightening, dyeing or bleaching compositions, or else in the form of rinse-out compositions, to be applied before or after dyeing, bleaching, permanent-waving or hair straightening, or else between the two steps of a permanent-waving or hair straightening procedure.

The compositions of the invention may also be in the form of washing compositions for the skin, such as in the form of bath or shower solutions or gels or of makeup-removing products.

The compositions according to the invention may also be in the form of aqueous or aqueous-alcoholic lotions for skincare and/or haircare.

The composition according to the invention has another advantageous application in the field of makeup, such as for making up the lips, the eyelashes and/or the face. The compositions may therefore advantageously be in the form of a makeup composition, such as mascara, eyeliner, lipstick, face powder, eyeshadow, or foundation.

A subject matter of the invention is also a method for the cosmetic treatment of keratin materials, such as the skin of the body or face, the lips, the nails, the hair and/or the eyelashes, comprising the application, to said materials, of a cosmetic composition as defined above.

This method according to the invention makes possible for example the form retention of the hairstyle or the treatment, care or washing of, or removal of makeup from, the skin, the hair or any other keratin material.

This method also makes it possible to make up the skin, the eyelashes, the nails, the hair, and/or the lips.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below

### EXAMPLE 1

The constituents of the vessel heel are introduced into a 1 liter reactor equipped with a condenser, a stirrer and a dropping funnel. Heating is carried out at 90° C. for 1 hour while stirring at 150 rpm. When the temperature reaches 90° C., an exothermicity of approximately 10° C. and whitening are observed. After a return to 90° C., the constituents of the portion run in are added over 1 hour. The temperature is maintained at 90° C. for 3 hours. The heptane is distilled under reduced pressure and is replaced with isododecane.

| | Vessel heel | Portion | Total |
|---|---|---|---|
| | (g) | run in (g) | amount |
| Methyl acrylate | 27 | 54 | 81 |
| MPEG 350* | — | 9 | 9 |
| Initiator (Trigonox 21S from Akzo)** | 1.35 | 0.99 | |
| Stabilizer (Kraton G1701) | 13.5 | — | |
| Isododecane | 270 | — | |
| Heptane | 103 | — | |

*MPEG 350: Methoxy poly(ethylene glycol) methacrylate, Bisomer MPEG from Cognis
**Trigonox 21S: Tert-butyl peroxy-2-ethylhexanoate.

A dispersion of particles of polymer (90% methyl acrylate +10% MPEG 350) in isododecane is obtained.

### EXAMPLE 2

In a manner similar to Example 1, the following polymer dispersion is prepared:

| | Vessel heel | Portion run | Total |
|---|---|---|---|
| | (g) | in (g) | amount |
| Methyl acrylate | 27 | 54 | 81 |
| MPEG 550* | — | 9 | 9 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

*MPEG 550: Methoxy poly(ethylene glycol) methacrylate available from Laporte; Bisomer MPEG from Cognis.

A dispersion of particles of polymer (90% methyl acrylate +10% MPEG 550) in isododecane is obtained.

### EXAMPLE 3

In a manner similar to Example 1, the following polymer dispersion is prepared:

| | Vessel heel | Portion run | Total |
|---|---|---|---|
| | (g) | in (g) | amount |
| Methyl acrylate | 22.5 | 54 | 76.5 |
| Acrylic acid | 4.5 | — | 4.5 |
| MPEG 550 | — | 9 | 9 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (85% methyl acrylate + 5% acrylic acid + 10% MPEG 550) in isododecane is obtained.

### EXAMPLE 4

In a manner similar to Example 1 the following polymer dispersion is prepared:

| | Vessel heel | Portion run | Total |
|---|---|---|---|
| | (g) | in (g) | amount |
| Methyl acrylate | 22.5 | 40.5 | 63 |
| Acrylic acid | 4.5 | — | 4.5 |
| MPEG 550 | — | 9 | 9 |
| Methyl methacrylate | — | 13.5 | 13.5 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (70% methyl acrylate + 5% acrylic acid + 15% methyl methacrylate + 10% MPEG 550) in isododecane is obtained.

### EXAMPLE 5

In a manner similar to Example 1, the following polymer dispersion is prepared:

| | Vessel heel | Portion run | Total |
|---|---|---|---|
| | (g) | in (g) | amount |
| Methyl acrylate | 27 | 36 | 63 |
| MPEG 350* | — | 27 | 27 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (70% methyl acrylate + 30% MPEG 350) in isododecane is obtained.

### EXAMPLE 6

In a manner similar to Example 1, the following polymer dispersion is prepared:

| | Vessel heel | Portion run | Total |
|---|---|---|---|
| | (g) | in (g) | amount |
| Methyl acrylate | 27 | 36 | 63 |
| MPEG 550* | — | 27 | 27 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (70% methyl acrylate + 30% MPEG 550) in isododecane is obtained.

### EXAMPLE 7

In a manner similar to Example 1 the following polymer dispersion is prepared:

| | Vessel heel | Portion run | Total |
|---|---|---|---|
| | (g) | in (g) | amount |
| Methyl acrylate | 22.5 | 36 | 58.5 |
| Acrylic acid | 4.5 | — | 4.5 |
| MPEG 550 | — | 27 | 27 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (65% methyl acrylate + 5% acrylic acid + 30% MPEG 550) in isododecane is obtained.

### EXAMPLE 8

In a manner similar to Example 1, the following polymer dispersion is prepared:

| | Vessel heel | Portion run | Total |
|---|---|---|---|
| | (g) | in (g) | amount |
| Methyl acrylate | 22.5 | 16.2 | 38.7 |
| Acrylic acid | 4.5 | — | 4.5 |
| MPEG 550 | — | 27 | 27 |
| Methyl methacrylate | — | 19.8 | 19.8 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (43% methyl acrylate + 5% acrylic acid + 22% methyl methacrylate + 30% MPEG 550) in isododecane is obtained.

### EXAMPLE 9

In a manner similar to Example 1, the following polymer dispersion is prepared:

| | Vessel heel | Portion run | Total |
|---|---|---|---|
| | (g) | in (g) | amount |
| Methyl acrylate | 22.5 | 49.5 | 72 |
| Acrylic acid | 4.5 | 4.5 | 9 |
| MPEG 550 | — | 9 | 9 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (80% methyl acrylate + 10% acrylic acid + 10% MPEG 550) in isododecane is obtained.

### EXAMPLE 10

In a manner similar to Example 1, the following polymer dispersion is prepared:

| | Vessel heel | Portion run | Total |
|---|---|---|---|
| | (g) | in (g) | amount |
| Methyl acrylate | 22.5 | 31.5 | 54 |
| Acrylic acid | 4.5 | 4.5 | 9 |
| MPEG 550 | — | 27 | 27 |
| Initiator (Trigonox 21S from Akzo) | 1.35 | 0.99 | |
| Stabilizer (Kraton G1701) | 13.5 | — | |
| Isododecane | 270 | — | |
| Heptane | 103 | — | |

A dispersion of particles of polymer (60% methyl acrylate + 10% acrylic acid + 30% MPEG 550) in isododecane is obtained.

### EXAMPLE 11

### 1/Preparation of the Comparative Example: Dispersion of Methyl Acrylate/Acrylic Acid 95/5% by Weight

The synthesis is carried out in semi-batch mode in an automated 1 liter reactor; the following are first of all introduced into the vessel heel:
22.5 g of methyl acrylate (MeA)
4.5 g of acrylic acid (AA)
1.35 g of Trigonox 21 S
13.5 g of Kraton G1701
270 g of isododecane
103 g of heptane

The reaction medium is heated to 90° C. in one hour, while at the same time stirring at 150 rpm. When the temperature of the reaction medium reaches 90° C., an exothermicity of approximately 10° C. and whitening are observed. Once the temperature has returned to 90° C., the introduction of the run-in portion is begun, and lasts approximately one hour. The latter contains:
63 g of methyl acrylate (MeA)
0.99 g of Trigonox 21S

Once the introduction of the run-in portion has been completed, polymerization is allowed to occur for 3 hours at 90° C. The heptane is subsequently distilled, under reduced pressure, and is replaced with isododecane.

### 2/Evaluation in a Wet Medium

Once films of polymer of approximately 1 mm wet thickness have been prepared (from a dispersion containing 20% by weight of polymer) on Teflon and cut up into squares (3×3 cm), they are immersed in water at 25° C. (0.2 g of polymer film in 20 g of water) and the change in color, in tack, in glide and in increase in mass from water uptake of these films are followed qualitatively. The following results are obtained (% by weight):
initially, on dry film (before immersion)
after 30 minutes of immersion
after 14 days of immersion
after 14 days of immersion+drying for 24 hours (on dry film

| Film | | Comparative | Ex. 1 | Ex. 2 | Ex. 3 | Ex. 4 |
|---|---|---|---|---|---|---|
| Initial (dry | Color | Bluish translucent | | | | |
| film) | Tack | 0 | | | | |
| | Glide | 0 | | | | |
| Color (after 30 min | | Slightly | White | White | White | White |
| immersion) | | cloudy | | | | |
| Immersed in | Color | Cloudy | White | White | White | White |
| water, 14 | Tack (with | 0 | | | | |
| days | the finger) | | | | | |
| | Glide (with | 0 | 0 | + | + | 0 |
| | the finger) | | | | | |
| | Increase in | 0 | 0 | 0 | + | 0 |
| | mass from | | | | | |
| | uptake | | | | | |
| | (weighed) | | | | | |

| Dry film: 24 h | Color | Cloudy translucent | | | | |
|---|---|---|---|---|---|---|
| drying | Tack | 0 | | | | |
| | Glide | 0 | | | | |
| Film | | Comparative | Ex. 5 | Ex. 6 | Ex. 7 | Ex. 8 |
| Initial (dry | Color | Bluish translucent | | | | Translucent |
| film) | Tack | 0 | + | ++ | ++ | 0 |
| | Glide | 0 | | | | |
| Color (30 min after | | Slightly | White | White | White | White |
| immersion) | | cloudy | | | | |
| Immersed in | Color | Cloudy | White | White | White | White |
| water (14 | Tack (with | 0 | | | | |
| days) | the finger) | | | | | |
| | Glide (with | 0 | + | + | ++ | 0 |
| | the finger) | | | | | |
| | Increase in | 0 | 0 | 0 | ++ | + |
| | mass from | | | | | |
| | uptake | | | | | |
| | (weighed) | | | | | |
| Dry film: 24 h | Color | Cloudy translucent | | | | |
| drying | Tack | 0 | + | + | + | 0 |

| | |
|---|---|
| Glide | 0 |

Tack: “0 = non-tacky”, “+ = slightly tacky”, “++ = quite tacky”
Glide: “0 = no glide”, “+ = slight glide”, “++ = quite a lot of glide”
Increase in mass from uptake: “0 = none”, “+ = slight”, “++ = quite a lot”

It is noted that the comparative polymer film is initially non-tacky. In a wet medium, it does not exhibit any glide, nor is it tacky, and neither whitening nor increase in mass from uptake are observed.

The films obtained with the dispersions according to the invention exhibit a high degree of glide in a wet medium, and will therefore be more comfortable to wear. Moreover, some films can absorb/take up water (they increase in mass) and swelling of the film is then observed, hence a greater volume.

### EXAMPLE 12

### Mascara

A mascara having the following composition is prepared:

| Waxes | 25 | g |
|---|---|---|
| 2-Amino-2-methyl-1,3-propanediol | 0.2 | g |
| Triethanolamine | 2.4 | g |
| Stearic acid | 5.4 | g |
| Dimethicone copolyol (Q2-5220 from Dow Corning) | 0.2 | g |
| Sodium polymethacrylate | 0.25 | g AM |
| Dispersion of Example 1 | 0.5 | g |
| Pigments | 6 | g |
| Preservatives | qs | |
| Water | qs 100 | g |

### EXAMPLE 13

### Body Care Oil

A moisturizing oil for dry skin is prepared using the following constituents:

| Caprylic/capric triglycerides | 6.5 | g |
|---|---|---|
| Propylene glycol dicaprylate/dicaprate | 22 | g |
| Cetearyl octanoate and isopropyl myristate | 5 | g |
| Isostearyl neopentanoate | 2.5 | g |
| Groundnut oil | 5.25 | g |
| Dispersion of Example 2 | 0.5 | g |
| Palmitic acid | 0.2 | g |
| Antioxidant, preservatives, fragrance | qs | |
| Cyclomethicone | qs 100 | g |

### EXAMPLE 14

### Moisturizing Cosmetic Composition

A moisturizing composition is prepared, comprising:

### Phase A1:

| | |
|---|---|
| Sucrose distearate | 2.0% |
| Oxyethylenated sorbitan stearate (4EO) | 1.4% |
| Stearic acid | 0.75% |
| Stearyl heptanoate | 5.5% |
| Codex petroleum jelly | 2.1% |
| Avocado oil | 4.5% |
| Jojoba oil | 4.1% |
| Volatile silicone oil | 3.7% |
| Dispersion of Example 3 | 1% |

### Phase A2:

| | |
|---|---|
| Silicone gum (Q2-1403 Fluid) | 4.0% |
| Fragrance, preservative | qs |

### Phase B:

| | |
|---|---|
| Preservative | qs |
| Triethanolamine | 0.4% |
| Water | qs 100% |

### Phase C:

| | |
|---|---|
| Carboxyvinyl polymers (Carbopol 980) | 0.3% |
| Water | 9.7% |

Phases A1 and B are brought to 65° C. before being combined (B in A1) with stirring (rotor-stator). Phase A2 is dispersed, at ambient temperature, in the first dispersion, with stirring. Phase C, prepared beforehand, is subsequently dispersed in order to gel the suspension. A moisturizing emulsion suitable for dry skin is obtained.

### EXAMPLE 15

### Lipstick Wand

The following lipstick composition is prepared (% by weight):

| | |
|---|---|
| Polyethylene wax | 15% |
| Dispersion of polymer of Example 3 | 10% of solids |
| Non-volatile carbon-based oil (Parleam) | 26% |
| Pigments | 8.6% |
| Isododecane | qs 100% |

The composition obtained after application to the lips exhibits good cosmetic properties.

### EXAMPLE 16

### Lipstick Wand

The following lipstick composition is prepared (% by weight):

| | |
|---|---|
| Polyethylene wax | 15% |
| Dispersion of polymer of Example 8 | 8% of solids |
| Non-volatile carbon-based oil (Parleam) | 26% |
| Pigments | 8.6% |
| Isododecane | qs 100% |

The composition obtained after application to the lips exhibits good cosmetic properties.

### EXAMPLE 17

### W/O Foundation

A foundation composition comprising the following compounds is prepared:

### Phase A

| | | |
|---|---|---|
| Cetyl dimethicone copolyol (Abil EM90) | 3 | g |
| Isostearyl diglyceryl succinate | 0.6 | g |
| (Imwitor 780K, company Condea) | | |
| Pigments | 10 | g |
| Isododecane | 18.5 | g |
| Dispersion of polymer of Example 3 | 8 | g of solids |

### Phase B

| Magnesium sulphate | 0.7 | g |
|---|---|---|
| Preservative | qs | |
| Water | qs 100 | g |

### Phase C

| | |
|---|---|
| Preservative | qs |
| Water | 2 g |

The composition obtained exhibits goods cosmetic properties.

What is claimed is:

1. Dispersion of particles of at least one ethylenic polymer surface-stabilized with at least one stabilizing agent, in a nonaqueous medium comprising at least one nonaqueous compound, which is liquid at 25° C. in which said nonaqueous medium has an overall solubility parameter according to the Hansen solubility space of less than or equal to 20 (MPa)^{1/2}
characterized in that said at least one ethylenic polymer comprises from 5% to less than 50% by weight of at least one monomer of formula (I), relative to the total weight of monomers: 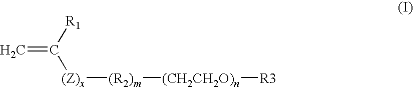
in which:
R1 is a hydrogen atom or a linear or branched CₚH₂ₚ₊₁ hydrocarbon-based radical, with p being an integer from 1 to 12;
Z is a divalent group chosen from —COO—, —CONH—, —CONCH₃—, —OCO—, —O—, —SO₂—, —CO—O—CO—, and —CO—CH₂—CO—;
x is 0 or 1;
R2 is a linear, branched, cyclic, optionally aromatic, or alicyclic, optionally aromatic, saturated or unsaturated, substituted or unsubstituted, divalent carbon-based radical containing from 1 to 30 carbon atoms, which may comprise from 1 to 18 heteroatoms chosen from O, N, S, F, Si, and P;
m is 0 or 1;
n is an integer from 3 to 300;
R3 is a hydrogen atom or a linear, branched, cyclic, optionally aromatic, or alicyclic, optionally aromatic, saturated or unsaturated, unsubstituted or substituted, carbon-based radical containing from 1 to 30 carbon atoms, which may comprise 1 to 20 heteroatoms chosen from O, N, S, F, Si, and P;
and/or at least one salt thereof.

2. Dispersion according to claim 1, in which Z represents —COO— or —CONH—.

3. Dispersion according to claim 2, in which Z represents —COO—.

4. Dispersion according to claim 1, in which R2 is:
an alkylene radical containing 1 to 20 carbon atoms;
a substituted or unsubstituted cycloalkylene radical containing 5 to 10 carbon atoms;
a phenylene radical —C₆H₄-(ortho, meta or para) unsubstituted or substituted with a C₁-C₁₂ alkyl radical optionally comprising 1 to 18 heteroatoms chosen from O, N, S, F, Si, and P;
a benzylene radical —C₆H₄—CH₂— unsubstituted or substituted with a C₁-C₁₂ alkyl radical optionally comprising 1 to 18 heteroatoms chosen from O, N, S, F, Si, and P;
a radical of formula —CH₂—O—CO—O—, —CH₂—CH₂—O—CO—O—, —CH₂—CO—O—, —CH₂—CH₂—CO—O—, —CH₂—O—CO—NH—, —CH₂—CH₂—O—CO—NH—; —CH₂—NH—CO—NH—, —CH₂—CH₂—NH—CO—NH—; —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—OH₂—CH₂—NR′—, —CH₂—CH₂—CH₂—O—; —CH₂—CH₂—CHR′—O— with R′ and R″ representing a linear or branched C₁-C₂₂ alkyl optionally comprising 1 to 12 heteroatoms chosen from O, N, S, F, Si, and P;
or a mixture of these radicals.

5. Dispersion according to claim 1, in which R3 is a hydrogen atom; a phenyl radical unsubstituted or substituted with a C₁-C₁₂ alkyl radical which may comprise 1 to 20 heteroatoms chosen from O, N, S, F, Si, and P; a C₁-C₃₀ alkyl radical which may comprise 1 to 18 heteroatoms chosen from O, N, S, F, Si, and P; or a C₃-C₁₂ cycloalkyl radical which may comprise 1 to 18 heteroatoms chosen from O, N, S, F, Si, and P.

6. Dispersion according to claim 1, in which the at least one monomer of formula (I) is such that:
R1=a hydrogen atom or methyl,
Z represents —COO—,
x=1
m=0,
n=from 7 to 100,
R3 is chosen from a hydrogen atom; a phenyl radical optionally substituted with a C₁-C₁₂ alkyl radical; and a C₁-C₃₀ alkyl radical,

7. Dispersion according to claim 6, in which the R3 is chosen from a C₁-C₂₂ alkyl radical.

8. Dispersion according to claim 6, in which the R3 is chosen from a C₂-C₁₆ alkyl radical.

9. Dispersion according to claim 1, in which the at least one monomer of formula (I) is chosen from:
poly(ethylene glycol) (meth)acrylate in which R1 is H or methyl, Z is —COO—, x=1, m=0, and R3=H;
methyl poly(ethylene glycol) (meth)acrylate in which R1 is H or methyl, Z is —COO—, x=1, m=0, and R3=methyl;
alkyl poly(ethylene glycol) (meth)acrylate in which R1 is H or methyl, Z is —COO—, x=1, m=0, and R3=alkyl;
poly(ethylene glycol) phenyl ether (meth)acrylate, in which R1 is H or methyl, Z is —COO—, x=l, m=0, and R3=phenyl.

10. Dispersion according to claim 1, in which the at least one monomer of formula (I) is chosen from poly(ethylene glycol) (meth)acrylates and alkyl poly(ethylene glycol) (meth)acrylates.

11. Dispersion according to claim 1, in which the at least one monomer of formula (I) is chosen from methyl poly(ethylene glycol) methacrylates.

12. Dispersion according to claim 1, in which the at least one ethylenic polymer comprises from 7% to 49% by weight, of monomer of formula (I) relative to the total weight of monomers.

13. Dispersion according to claim 1, in which the at least one ethylenic polymer comprises from 8% to 45% by weight, of monomer of formula (I) relative to the total weight of monomers.

14. Dispersion according to claim 1, in which the at least one ethylenic polymer comprises from 10% to 40% by weight of monomer of formula (I) relative to the total weight of monomers.

15. Dispersion according to claim 1, in which the at least one ethylenic polymer comprises from 15% to 35% by weight, of monomer of formula (I) relative to the total weight of monomers.

16. Dispersion according to claim 1, in which the at least one ethylenic polymer comprises from 50% to 95% by weight, relative to the total weight of monomers, of at least one additional monomer chosen from:
(i) the monomers of formula (II), and salts thereof: 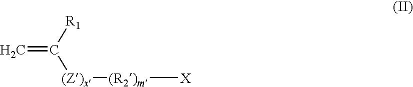
in which:
R1 is a hydrogen atom or a linear or branched CₚH₂ₚ₊₁ hydrocarbon-based radical, with p being an integer from 1 to 12;
Z′ is a divalent group chosen from —COO— and —CONH—;
x′ is 0 or 1;
R2′ is a linear, branched, cyclic, optionally aromatic, or alicyclic, optionally aromatic, saturated or unsaturated, divalent carbon-based radical containing from 1 to 30 carbon atoms, which may comprise 1 to 18 heteroatoms chosen from O, N, S, F, Si, and P;
m′ is 0 or 1;
X is chosen from —H, —COOH, —SO₃H, —OSO₃H, —PO₃H₂, and —OPO₃H₂;
(ii) (meth)acrylic acid esters of formula CH₂═CHCOOR4 or CH₂═C(CH₃)COOR4 with R4 representing a linear, cyclic or branched, saturated or unsaturated, hydrocarbon-based radical, containing 1 to 32 carbon atoms, optionally comprising intercalated at least one heteroatom chosen from O, N, and S, and said hydrocarbon based radical being unsubstituted or substituted with at least one substituent chosen from —OH and halogen atoms chosen from Cl, Br, I, and F;
(iii) (meth)acrylamides of formula CH₂═CHCONR5R15 or CH₂═C(CH₃)CONR5R15 in which R5 and R15, which may be identical or different, represent a hydrogen atom or a linear, cyclic or branched, saturated or unsaturated, optionally aromatic, hydrocarbon-based radical containing 7 to 28 carbon atoms, optionally comprising intercalated at least one heteroatom chosen from O, N, and S, and said hydrocarbon based radical being unsubstituted or substituted with at least one substituent chosen from —OH and halogen atoms chosen from Cl, Br, I, and F;
(iv) vinyl esters of formula CH₂═CH—OCO—R6 with R6 representing a linear or branched, saturated or unsaturated, hydrocarbon-based radical, containing 1 to 12 carbon atoms;
(v) vinyl ethers of formula CH₂═CHOR7 with R7 representing a linear or branched, saturated or unsaturated, hydrocarbon-based radical, containing 2 to 12 carbon atoms;
(vi) vinyl compounds of formula CH₂═CHR8 in which R8 is:
a hydroxyl group;
a linear or branched alkyl group containing 1 to 25 carbon atoms, in which is optionally intercalated at least one heteroatom chosen from O, N, S, and P; it being possible for said alkyl group, in addition, to be unsubstituted or substituted with at least one substituents chosen from —OH and halogen atoms chosen from Cl, Br, I, and F;
a C₃ to C₈ cycloalkyl group;
a C₆ to C₂₀ aryl group;
a C₇ to C₃₀ aralkyl group (C₁ to C₄ alkyl group);
a heterocyclic group comprising 4 to 12 ring members containing at least one heteroatom chosen from O, N, and S;
a heterocycloalkyl group (alkyl containing 1 to 4 carbon atoms);
it being possible for said cycloalkyl, aryl, aralkyl, heterocyclic, and heterocycloalkyl groups to be optionally unsubstituted or substituted with at least one substituent chosen from the hydroxyl group, halogen atoms, and linear or branched C₁-C₄ alkyl groups in which are optionally intercalated at least one heteroatom chosen from O, N, S, and P, it being possible, in addition, for said alkyl groups to be optionally substituted with at least one substituent chosen from —OH and halogen atoms chosen from Cl, Br, I, and F.

17. Dispersion according to claim 16,
in which said x′ is 1,
in which said C₃ to C₈ cycloalkyl group is a cyclohexane,
in which said C₆ to C₂₀ aryl group is a phenyl,
in which said C₇ to C₃₀ aralkyl group is chosen from 2-phenylethyl and benzyl,
in which said heterocycloalkyl group is chosen from furfuryl, furfurylmethyl, and tetrahydrofurfurylmethyl.

18. Dispersion according to claim 16, in which the at least one additional monomer is chosen from:
the (meth)acrylic acid esters with R4 as C₁-C₁₈;
styrene, ethyl hexyl vinyl ether, dodecyl vinyl ether, and vinyl hexanoate;
acrylic acid and methacrylic acid.

19. Dispersion according to claim 18, in which the (meth)acrylic acid esters with R4 as C₁-C₁₈, are chosen from methyl(meth)acrylate, ethyl(meth)acrylate, propyl (meth)acrylate, n-butyl(meth)acrylate, isobutyl(meth)acrylate, 2-ethylhexyl (meth)acrylate, tert-butyl(meth)acrylate, isooctyl(meth)acrylate, decyl (meth)acrylate, myristyl (meth)acrylate, stearyl(meth)acrylate, and isobornyl(meth)acrylate;

20. Dispersion according claim 1, in which the at least one nonaqueous liquid compound is chosen from:
plant oils formed by fatty acid esters of polyols;
hydrocarbons;
silicone oils, unsubstituted or substituted, with optionally fluorinated aliphatic and/or aromatic groups, and/or with functional groups, and optionally volatile;
solvents, alone or as a mixture, chosen from linear, branched, and cyclic esters containing 6 to 30 carbon atoms, ethers containing 6 to 30 carbon atoms, and ketones containing 6 to 30 carbon atoms;
aliphatic fatty monoalcohols containing at least 6 carbon atoms, the hydrocarbon-based chain of said aliphatic fatty monoalcohols not comprising a substituting group.

21. Dispersion according claim 20,
in which the said plant oils is a triglyceride;
in which the said hydrocarbons is chosen from paraffin oil, liquid petroleum jelly, hydrogenated polyisobutylene, C₈-C₁₆ isoparaffins, and volatile isoparaffins;
in which the said silicone oils are substituted with at least one functional group chosen from hydroxyl, thiol, and amine group;
in which the said silicone oils are volatile and chosen from cyclic and linear silicone oils;
in which the said aliphatic fatty monoalcohols contain 6 to 32 carbon atoms.

22. Dispersion according claim 20,
in which the said triglyceride is chosen from sunflower oil, sesame oil, rapeseed oil, and esters derived from 6 to 20 carbon atoms acids and alcohols;
in which the hydrocarbons are chosen from isododecane and “ISOPARs”;
in which the silicone oils are chosen from polydimethylsiloxanes and polymethylphenylsiloxanes;
in which the silicone oils are volatile and are chosen from cyclodimethylsiloxanes, cyclophenylmethylsiloxanes, and linear dimethylsiloxanes.

23. Dispersion according to claim 20, in which the silicone oils are chosen from linear dodecamethylpentasiloxane (L5), octamethylcyclotetrasiloxane, decamethylcyclopentasiloxane, hexadecamethylcyclohexasiloxane, heptamethylhexyltrisiloxane, and heptamethyloctyltrisiloxane.

24. Dispersion according to claim 1, in which the at least one nonaqueous liquid compound is chosen from esters derived from 6 to 20 carbon atoms acids and alcohols with a formula RCOOR′ in which R represents the residue of a fatty acid containing from 7 to 19 carbon atoms and R′ represents a hydrocarbon-based chain containing from 3 to 20 carbon atoms.

25. Dispersion according to claim 1,
in which the at least one nonaqueous liquid compound ester is chosen from palmitates, adipates, myristates, and benzoates.

26. Dispersion according to claim 1, in which the at least one nonaqueous liquid compound ester is chosen from diisopropyl adipate and isopropyl myristate.

27. Dispersion according to claim 1, in which the at least one stabilizing agent is chosen from sequential polymers, grafted polymers, and random polymers.

28. Dispersion according to claim 1, in which the at least one stabilizing agent is present in a proportion from 0.1% to 30% by weight relative to the total weight of monomers.

29. Dispersion according to claim 1, in which the at least one stabilizing agent is present in a proportion from 1% to 20% by weight relative to the total weight of monomers.

30. Dispersion according to claim 1, in which the at least one stabilizing agent is present in a proportion from 2% to 15% by weight relative to the total weight of monomers.

31. Dispersion according to claim 1, in which the at least one stabilizing agent is present in a proportion from 3% to 10% by weight relative to the total weight of monomers.

32. Dispersion according to claim 1, in which the at least one stabilizing agent is chosen from:
silicone polymers grafted with a hydrocarbon-based chain and hydrocarbon-based polymers grafted with a silicone chain;
grafted copolymers having an insoluble backbone of polyacrylic type with soluble grafts of poly(12-hydroxystearic acid) type;
grafted- and sequential-block copolymers comprising at least one block of polyorganosiloxane and at least one block of a polymer (i) resulting from radical polymerization or (ii) resulting from polycondensation, it being possible for said copolymers to comprise fluorinated entities;
grafted- and sequential-block copolymers chosen from C₁-C₄ alkyl (meth)acrylates and C₈-C₃₀ alkyl (meth)acrylates;
grafted- and sequential-block copolymers comprising at least one block resulting from the polymerization of ethylenic monomer, comprising at least one optionally conjugated ethylenic bond; and at least one block of polymer resulting from radical polymerization other than from dienes;
alkyl dimethicones in which the alkyl group comprises 6 to 32 carbon atoms;
dimethiconol esters of formula: 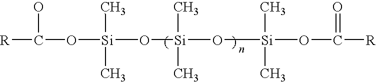
in which R is an alkyl radical containing 6 to 32 carbon atoms;
alkylamidoamines;
copolymers represented by the formula: 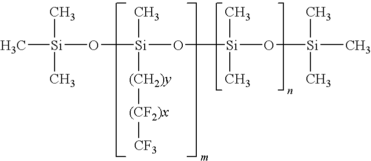
in which x is an integer from 3 to 12; y is an integer from 2 to 6; and m and n are such that the molecular weight of the compound is from 5000 to 15 000.

33. Dispersion according to claim 1,
in which the at least one stabilizing agent is chosen from grafted- and sequential-block copolymers comprising at least one block of polyorganosiloxane and at least one block of a polymer chosen from polyethers, polyesters, polyamides, and blends thereof, it being possible for said copolymers to comprise fluorinated entities;
in which said ethylenic monomer is chosen from dienes;
in which the at least one block of polymer resulting from radical polymerization other from dienes is chosen from vinyl monomers, (meth)acrylic monomers, (meth)acrylamide monomers, polyethers, polyesters, polyamides, and blends thereof;
in which said alkyl dimethicones are chosen from lauryl methicone, stearyl methicone, cetyl dimethicone, and behenoxy dimethicone;
in which said dimethiconol esters are chosen from dimethiconol behenate;
in which said alkylamidoamines contain 6 to 60 carbon atoms;
in which x is an integer from 5 to 10;
in which y is an integer from 2 to 3.

34. Dispersion according to claim 33,
in which alkylamidoamines contain 12 to 50 carbon atoms;
in which x=8.

35. Dispersion according to claim 33, in which the alkylamidoamines are chosen from behenamidopropyldimethylamine.

36. Cosmetic or pharmaceutical composition comprising, in a cosmetically or pharmaceutically acceptable medium, at least one dispersion of particles of at least one ethylenic polymer surface-stabilized with at least one stabilizing agent, in a nonaqueous medium comprising at least one nonaqueous compound, which is liquid at 25° C., having an overall solubility parameter according to the Hansen solubility space of less than or equal to 20 (MPa)^{1/2}
characterized in that said at least one ethylenic polymer comprises from 5% to less than 50% by weight of at least one monomer of formula (I), relative to the total weight of monomers: 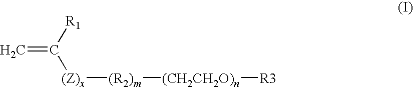
in which:
R1 is a hydrogen atom or a linear or branched CₚH₂ₚ₊₁ hydrocarbon-based radical, with p being an integer from 1 to 12;
Z is a divalent group chosen from —COO—, —CONH—, —CONCH₃—, —OCO—, —O—, —SO₂—-CO—O—CO— and —CO—CH₂—CO—;
x is 0 or 1;
R2 is a linear, branched, cyclic, optionally aromatic, or alicyclic, optionally aromatic, saturated or unsaturated, substituted or unsubstituted, divalent carbon-based radical containing from 1 to 30 carbon atoms, which may comprise from 1 to 18 heteroatoms chosen from O, N, S, F, Si, and P;
m is 0 or 1;
n is an integer from 3 to 300;
R3 is a hydrogen atom or a linear, branched, cyclic, optionally aromatic, or alicyclic, optionally aromatic, saturated or unsaturated, unsubstituted or substituted, carbon-based radical containing from 1 to 30 carbon atoms, which may comprise 1 to 20 heteroatoms chosen from O, N, S, F, Si, and P;
and/or at least one salt thereof.

37. Composition according to claim 36, furthermore comprising at least one constituent chosen from waxes, oils, gums and pasty fatty substances of plant, animal, mineral, and synthetic origin, and silicone origin; dyestuffs chosen from pulverulent compounds, and fat-soluble, and water-soluble dyes; antioxidants, fragrances, essential oils, preservatives, cosmetic active agents, moisturizing agents, vitamins, essential fatty acids, ceramides, sunscreens, surfactants, polymers, thickeners, and gelling agents.

38. Composition according to claim 36, which is in the form of a product for caring for and/or making up the skin of the body or face, the lips and the hair, of an antisun or self-tanning product, or of a hair product.

39. Composition according to claim 38, in which the said hair product is chosen from the products for the treatment, the washing, the form retention, and the shaping of the hair.

40. Composition according to claim 36, which is in the form of a shampoo, gel, hair-setting lotion, blow-drying lotion, or fixing and styling composition, of a rinse-out or leave-in conditioner, of a permanent-waving, hair straightening, dyeing or bleaching composition, or of a rinse-out composition, to be applied before or after dyeing, bleaching, permanent-waving, or hair straightening, or else between the two steps of a permanent-waving or hair straightening procedure; of washing compositions for the skin, or of aqueous, or aqueous-alcoholic lotions for skincare, and/or haircare.

41. Composition according to claim 40,
in which the fixing and styling composition is chosen from lacquers, mousses, and sprays;
in which the washing compositions for the skin are chosen from the form of bath or shower solutions or gels.

42. Composition according to claim 36, which is in the form of a composition for making up the lips, the eyelashes and/or the face.

43. Composition according to claim 36, which is in the form of a composition chosen from mascaras, eyeliners, lipsticks, face powders, eyeshadows, and foundations.

44. Method for the cosmetic treatment of keratin materials, comprising applying to said materials, a cosmetic composition comprising at least one dispersion of particles of at least one ethylenic polymer surface-stabilized with at least one stabilizing agent, in a nonaqueous medium comprising at least one nonaqueous compound, which is liquid at 25° C., having an overall solubility parameter according to the Hansen solubility space of less than or equal to 20 (MPa)^{1/2}
characterized in that said at least one ethylenic polymer comprises from 5% to less than 50% by weight of at least one monomer of formula (I), relative to the total weight of monomers: 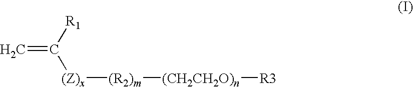
in which:
R1 is a hydrogen atom or a linear or branched CₚH₂ₚ₊₁ hydrocarbon-based radical, with p being an integer from 1 to 12;
Z is a divalent group chosen from —COO—, —CONH—, —CONCH₃—, —OCO—, —O—, —SO₂—-CO—O—CO— and —CO—CH₂—CO—;
x is 0 or 1;
R2 is a linear, branched, cyclic, optionally aromatic, or alicyclic, optionally aromatic, saturated or unsaturated, substituted or unsubstituted, divalent carbon-based radical containing from 1 to 30 carbon atoms, which may comprise from 1 to 18 heteroatoms chosen from O, N, S, F, Si, and P;
m is 0 or 1;
n is an integer from 3 to 300;
R3 is a hydrogen atom or a linear, branched, cyclic, optionally aromatic, or alicyclic, optionally aromatic, saturated or unsaturated, unsubstituted or substituted, carbon-based radical containing from 1 to 30 carbon atoms, which may comprise 1 to 20 heteroatoms chosen from O, N, S, F, Si, and P;
and/or at least one salt thereof.

45. Method for the cosmetic treatment of claim 44, in which the said keratin materials are chosen from the skin of the body or face, the lips, the nails, the hair, and/or the eyelashes.
